(19) United States
(12) Patent Application Publication
(10) Pub. No.: US 2024/0261117 A1
(43) Pub. Date: Aug. 8, 2024

(54) Method and System of Digital Design and Fabrication of a Biomechanical Interface

(71) Applicant: Massachusetts Institute of Technology, Cambridge, MA (US)

(72) Inventors: Hugh M. Herr, Concord, NH (US); Duncan Ru Chieh Lee, Cambridge, MA (US); Christina Meyer, Cambridge, MA (US); Dana Solav, Caesarea (IL); Xingbang Yang, Beijing (CN)

(21) Appl. No.: 18/527,057

(22) Filed: Dec. 1, 2023

Related U.S. Application Data
(60) Provisional application No. 63/429,871, filed on Dec. 2, 2022.

(51) Int. Cl. A61F 2/78

(52) U.S. Cl. CPC A61F 2/7812; A61F 2002/7818

(57) ABSTRACT

Aspects of inventive concepts are generally directed to a liner, methods of use, methods of design, and related systems. In various embodiments, the liner is configured to serve as an interface between an external surface of a biological body segment of a subject and a prosthetic socket, the biological body segment being amputated below a joint. In various embodiments, a pressure applied by the liner to the biological body segment varies over the length of the liner with a maximum pressure applied at the joint and a lesser pressure applied below the joint.

### RELATED APPLICATION

This application claims the benefit of U.S. Provisional Application No. 63/429,871, filed on Dec. 2, 2022. The entire teachings of the above application are incorporated herein by reference.

### GOVERNMENT SUPPORT

This invention was made with government support under 5R01EB024531-03 from the National Institutes of Health. The government has certain rights in the invention.

### BACKGROUND

Today there are more than 2 million people in the United States living with limb loss, with that number projected to grow to 3.6 million by 2050 [5, 6, 7]. People with disability are more likely to be at a socioeconomic disadvantage, show increased incidence of using emergency healthcare services, and express a worse outlook on overall health compared to those without disability [8]. Lower-extremity amputation is the most common form of amputation, with transtibial (below-knee) amputation making up a significant fraction [9]. Reasons for transtibial amputation range from trauma to dysvascular disease, and with the increasing rates of diabetes worldwide, the population of transtibial amputees will only increase in the future [10, 11]. Amputation has long-lasting physical and mental effects, including chronic residual limb pain, phantom pain, and skin sores, diminish quality of life, and exacerbation psychological problems.

The prosthetic interface, comprising of a liner and socket, is the main determinant in the comfort and wearability of a prosthetic device. The liner acts as a cushion between the skin and the socket, while the socket ensures that load is distributed across the limb in a comfortable manner. The socket geometry also provides stability and structure during prosthesis use. While mechanical components of a prosthesis may last for over 5 years, prosthetic sockets and liners must often be replaced every 1-3 years due to wear and the evolving shape of the residual limb. Conventional interface design has a prosthetist create a custom socket for a patient's limb. This involves covering the limb in plaster to make a negative mold, from which a positive mold is made to create a replica of the limb. A trained prosthetist hand modifies the positive mold using artisanal methods in an attempt to apply load in high load-bearing areas and to create relief in areas of high sensitivity. Following this modification process, the positive mold is used to make a check socket that is then tested by the patient and modified iteratively until the comfort is deemed acceptable by the patient [12]. This process requires multiple visits to the prosthetist and can take many weeks to achieve a comfortable fit. Conventional liners are off-the-shelf items that come in discrete sizes and therefore are not subject-specific in their design. For such an important part of a prosthesis, this design method is largely artisanal, without clear scientific rationale, is highly time and effort intensive, and is neither repeatable nor cost effective [13]. The quality and comfort of the final socket is also dependent on the skill of the prosthetist, and therefore there is inequity in the quality of prosthetic care for people of different income levels [14]. Improper fit of the prosthetic interface can lead to many injuries such as skin irritation, bursas, and ulcers. It can also cause long-term harmful changes to posture and gait. Poor fitting sockets make it more likely a person will not use their prosthesis altogether, and the reduction in mobility can lead to increased rates of obesity and other health complications [15, 16, 17].

More recently, prosthetic interface design has been augmented by the use of three-dimensional scanners and computer-aided design software. Companies such as ProsFit™ have demonstrated prosthetic socket design pipelines that utilize this technology [18]. Other 3D scanners on the market can also be used for this purpose. These scanners allow for the elimination of the first casting step in conventional socket design, and also allow for the creation and manipulation of a 3D geometrical model. However, this type of scanning only captures surface geometry and lacks any information about bone structure and soft tissue depth. These components are critical if loading simulation is performed on the model. Also, there are very few custom prosthetic liner services on the market, and no all-in-one custom full prosthetic interface design services.

There exists a need to improve the prosthetic interface design and fabrication process in a way that can fully account for the unique shapes and properties of each person's limb while simultaneously reducing the associated time and cost of the process.

### SUMMARY

Aspects of inventive concepts are directed generally to methods and systems for designing a biomechanical interface of a device contacting a biological body segment of a subject.

More specifically, this description details specific modification steps for computationally design of prosthetic sockets and liners.

A computational framework involves using non-invasive imaging techniques (for example, magnetic resonance imaging (MRI) or computed tomography (CT)) to reconstruct a three-dimensional biomechanical model of a residual limb (residuum). This model contains information on the bone structure and soft tissue depth of the limb. The prosthetic interface is then designed around the geometry of the model. Dimensions and material properties are applied to the prosthetic liner design and socket design, and numerical methods (for example, finite element analysis (FEA)) is used to simulate interface donning and loading steps in order to approximate limb deformation and determine accurate geometry for real-world use. The models can be customized based on subject-specific feedback and preferences, and every change can be saved in a framework unique to that subject. The interface models can then be prepared for fabrication through rapid processes such as 3D printing.

The described methods and devices address changes to the definition and orientation of the axes used to define the socket and liner preliminary geometry that is used as the input for analysis. Aligning the axes with the orientation of the bone structure allows for better subject-specific design and therefore improved comfort of the final socket. Changes to the preliminary geometry definition are discussed both in the context of individual variation as well as broadly applicable design changes.

Another aspect of the described methods and devices involves the non-uniform pressure mapping of a custom liner. Distinct regions of the liner are defined, with different pressures applied to each based on known geometry, pain, and load capabilities. These regions are by default distal, lower leg, knee, and upper leg. Both the region geometry and applied pressure values can be adjusted based on subject-specific preferences, with the modifications saved in the algorithm. The liner is the component of the prosthetic interface that is least tailored to the user in conventional interface design, and previous methods to customize liners either only capture external geometry and/or apply constant pressure across the limb. Varying the pressure applied to the limb by the liner increases the resolution of customization capability in liner design, creating the possibility of more unique solutions to subject-specific limb pain.

A detailed description of non-uniform socket pressure mapping along with default values used for new subject socket design is provided. The pressure map is generally divided into four regions: distal, fibular head, patellar tendon, and residual, with residual being everything outside the first three. Regions are based off bone and tendon geometry and pressures are determined by soft tissue depth and literature on the load bearing capacity of different parts of the limb. The socket pressure map can also be adjusted to accommodate any subject-specific areas of concern.

A method of finite element analysis control is described, called “force control”. In this method, the subject's bodyweight is specified and applied as a load to the prosthetic interface. The deformation of the limb and interface is calculated based on this loading scenario. This method is different from previously described “displacement control” methods in that it applies load and calculates displacement rather than the other way around. Force control provides a better simulation of loading because the subject's actual bodyweight is used, rather than applying displacement values to try to guess the loading weight.

This description also details an expansive range of manual modifications that can be applied to the socket and liner mesh models after finite element analysis donning and loading simulation. These include mesh node manipulation to relieve undo pressure at certain regions of the interface, separation of the socket into distal and proximal regions for differential smoothing, socket geometry correction, liner extrusion, and socket and liner offsetting for final fabrication models. This host of modification capabilities enables further customization of the prosthetic interface. After all necessary modifications are complete, the interface is prepared for 3D printing.

Aspects of inventive concepts involve the ability to vary the thickness of a custom liner based on skin strain data. Uniform thickness liners often elicit complaints from users, especially around the knee region where material can bunch up posteriorly during knee bending. The variable thickness algorithm described retains uniform thickness below the socket cutline where the liner interfaces with the socket but reduces the thickness of the liner above the cutline based on regions where the skin strains the most during knee bending.

The provided methods and devices can specify the ability to use 3D printing to fabricate both prosthetic liners and check sockets. 3D printing allows for rapid and accurate fabrication of the prosthetic interface at lower cost than traditional manufacturing methods such as thermoforming or casting. The ability to 3D print soft materials such as silicone drastically reduces the time and material waste involved in making custom liners, providing a marked benefit over conventional liners as well as other forms of custom liner design. Machines capable of 3D printing soft materials are still in relative infancy, and therefore accuracy, resolution, and tolerances on parts are not as good as they are for rigid materials. Therefore, the thickness of each liner is measured after it is received, and the actual thickness is input into the computational framework.

This description presents detailed modifications and updates to previously described computational prosthetic interface design frameworks, with every change aimed at improving the comfort of digitally designed sockets. Each change is based on quantitative analysis of the residual limb, and therefore can be made more accurately than the conventional method allows. All the modifications can be incorporated seamlessly into a computational framework and have been used to design and produce sockets for subjects to walk on. The improvement in detail and resolution of design as well as new manufacturing methods can reduce the time, expense, and waste of prosthetic interface design.

Aspects of inventive concepts are generally directed to a liner configured to serve as an interface between an external surface of a biological body segment of a subject and a prosthetic socket, the biological body segment being amputated below a joint, wherein pressure applied by the liner to the biological body segment varies over the length of the liner with a maximum pressure applied at the joint and a lesser pressure applied below the joint.

In various embodiments, the pressure applied by the liner is lower than at the joint both below and above the joint.

In various embodiments, the joint is a knee.

In various embodiments, the maximum pressure applied at the joint is greater than 97.5 kPa.

In various embodiments, the lesser pressure varies between 52.5 kPa and 97.5 kPa.

In various embodiments, the liner comprises a first thickness below a cutline and a second thickness above a cutline and the first thickness is greater than the second thickness.

In various embodiments, the second thickness is between 2 mm and 7 mm.

In various embodiments, the liner is fabricated using direct-write, soft material 3D printing.

In various embodiments, the liner is fabricated using direct-write soft material 3D printing from platinum-cure silicone or polyurethane.

Aspects of inventive concepts are generally directed to a method for designing a liner interfacing an external surface of a biological body segment amputated below a joint, the method comprising: with a computer, forming a liner design based, at least in part, on a model of the biological body segment; and with a computer, adjusting the liner design in response to a simulated fitting pressure, the simulated fitting pressure varying over the length of the liner design with a maximum simulated fitting pressure being applied at positions in the liner design configured to interface at the joint and a lesser simulated fitting pressure applied at positions in the liner design configured to interface below the joint.

In various embodiments, the simulated fitting pressure applied at positions in the liner design configured to interface above and below the joint is lower than the simulated fitting pressure applied at positions in the liner design configured to interface at the joint.

In various embodiments, the joint is a knee.

In various embodiments, the maximum simulated fitting pressure is greater than 97.5 kPa.

In various embodiments, the lesser simulated fitting pressure varies between 52.5 kPa and 97.5 kPa.

In various embodiments, the liner design comprises a first thickness below a cutline and a second thickness above a cutline and the first thickness is greater than the second thickness.

In various embodiments, the second thickness is between 2 mm and 7 mm.

In various embodiments, the method further comprises fabricating the liner based on the liner design using direct-write, soft material 3D printing.

In various embodiments, the method further comprising fabricating the liner based on the liner design using direct-write soft material 3D printing from platinum-cure silicone or polyurethane.

Aspects of inventive concepts are generally directed to a system for designing a liner interfacing an external surface of a biological body segment amputated below a joint, comprising: a computer configured to form a liner design based, at least in part, on a model of the biological body segment; and adjust the liner design in response to a simulated fitting pressure, the simulated fitting pressure varying over the length of the liner design with a maximum simulated fitting pressure being applied at positions in the liner design configured to interface at the joint and a lesser simulated fitting pressure applied at positions in the liner design configured to interface below the joint.

Aspects of inventive concepts are generally directed to a method for interfacing a liner with an external surface of a biological body segment amputated below a joint, comprising: applying a pressure by the liner to the biological body segment, the pressure varying over the length of the liner with a maximum pressure applied at the joint and a lesser pressure applied below the joint.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows an example embodiment of local axes defined by the principal directions of the tibia, in accordance with aspects of inventive concepts.

FIG. 2 shows medial and lateral views of an example embodiment of a socket cutline definition points and curve, in accordance with aspects of inventive concepts.

FIGS. 3A-3D show an example embodiment of ray tracing to establish position of points 1, 7, 8, and 9, in accordance with aspects of inventive concepts.

FIG. 4 shows a posterior view of a transtibial residual limb, with anatomical points i and j labeled as 1 and 2, respectively. Point i is the outer tibial condyle and point j is the inner tibial condyle.

FIG. 5 shows a separation of a limb into three distinct regions: proximal, cutline, socket, in accordance with aspects of inventive concepts.

FIG. 6 shows a section view of an example embodiment of a mesh used in finite element analysis, in accordance with aspects of inventive concepts.

FIG. 7A shows an example embodiment of liner fitting pressure regions at a liner mesh, in accordance with aspects of inventive concepts.

FIG. 7B shows an example embodiment of liner fitting pressure regions at a liner mesh with a socket mesh overlapping a portion of the liner mesh, in accordance with aspects of inventive concepts.

FIG. 8 shows an example embodiment of a liner measurement technique, in accordance with aspects of inventive concepts.

FIG. 9 shows an example of socket pressure regions and fibular head region foci, in accordance with aspects of inventive concepts.

FIG. 10 shows a tibia center line, in accordance with aspects of inventive concepts.

FIGS. 11A-11C show an example of socket adjustments made post-finite element analysis, including cutline correction, geometric offsetting, and unification of offset surfaces using a loft, in accordance with aspects of inventive concepts.

FIG. 12 shows an example embodiment of a full liner geometry, in accordance with aspects of inventive concepts.

FIG. 13A shows an example embodiment of a socket mesh, four points defined based on defined dimensional parameters, and a curve that defines the bottom edge of the foot based on the four points, in accordance with aspects of inventive concepts.

FIG. 13B shows an example embodiment of a socket with a defined surface that connects the bottom curve of the foot to the socket, in accordance with aspects of inventive concepts.

FIG. 14A shows an example embodiment of a mesh with a foot, in accordance with aspects of inventive concepts.

FIG. 14B shows an example embodiment of a mesh without a foot, in accordance with aspects of inventive concepts.

FIGS. 15A-15B show example embodiments of various types of manual enlargement methods to relieve socket pressure, in accordance with aspects of inventive concepts.

FIG. 16 shows an example embodiment of distal enlargement of a mesh to relieve distal tibia pressure, in accordance with aspects of inventive concepts.

FIGS. 17A-17B show an example embodiment of a mesh comprising separation of socket geometry into proximal, transitional, and distal regions, in accordance with aspect of inventive concepts.

FIG. 18 shows an example embodiment of a liner mesh comprising an extruded surface, in accordance with aspects of inventive concepts.

FIG. 19A shows example of calculated absolute strain values mapped on to the unprocessed DIC model of a limb, in accordance with aspects of inventive concepts.

FIG. 19B shows a calculated example of a variable liner thickness offset from the skin, with constant thickness below the socket cutline, in accordance with aspects of inventive concepts.

FIG. 20 shows an example embodiment of a socket positive mold for permanent socket fabrication, in accordance with aspects of inventive concepts.

### DETAILED DESCRIPTION

A description of example embodiments follows.

X-axis is the normal to the coronal plane, defining anterior/posterior position. Positive is the posterior direction.

Y-axis is the normal to the sagittal plane, defining medial/lateral position Positive is to the right when facing the anterior (negative X) direction.

Z-axis is the normal to the transverse plane, defining proximal/distal position Positive is the proximal direction.

Systems and methods to digitally design and fabricate a biomechanical interface from a reconstructed biomechanical model of a biological limb segment are discussed herein. In the case of an amputated residuum as the biological limb segment, the preliminary interface geometry is designed based on residuum bone structure and soft tissue, and the entire model is converted into a mesh for numerical analysis (for example, using finite element analysis (FEA)). Non-uniform pressure maps are assigned to both the liner and socket components of the interface, representing the goals for convergence of FEA. The process of FEA simulates donning and loading of the biomechanical interface by applying appropriate bodyweight load and calculating pressure and displacement. Further subject-specific modifications that are found through subject fitting and testing but cannot be addressed by FEA are applied before the final interface geometry is converted to a 3D-printing ready model. Both prosthetic liner and socket are fabricated by 3D printing and arrive ready-to-wear.

In many of the embodiments discussed herein, finite element analysis is used. In alternative embodiments, a different numerical analysis approach may be used.

In various embodiments, the first step of the computational framework is the imaging of the biological body part for which the biomechanical interface is to be designed. Imaging techniques include, but are not limited to, magnetic resonance imaging (MRI), traditional computed tomography (CT), cone-beam computed tomography (CBCT), digital image correlation (DIC), and ultrasound (US), to name a few. There are advantages and disadvantages of each imaging technique. For example, MRI scans give more detail of the soft tissues within the residuum compared with CBCT. However, CBCT is faster, less expensive, and the machines can be smaller in footprint. The computational framework presented herein has the capability to process and construct a biomechanical model from any of these non-contact imaging modalities.

In the following section, we provide an example of the amputated transtibial residuum, but it should be understood that the same techniques can be applied to the design of any biomechanical interface that connects a biological segment to a designed interface. Further, in this example, the imaging tools of MRI and DIC are employed in the biomechanical data collection, but it should be understood that other imaging tools could be employed (e.g., CBCT) to build the biomechanical model of the biological segment.

In the case of a transtibial amputation, in the example presented herein, the residuum is first imaged using MRI. After the residuum is imaged, the image slices are segmented into corresponding anatomical structures using segmenting software (e.g., the GIBBON toolbox [19]). In some embodiments, these structures are: skin, femur, tibia, fibula, patella, patellar tendon, and any locating MRI markers. A contour is generated for each image slice, and the contours are combined into a complete levelset once every slice has been completed. The levelsets are then meshed into three-dimensional surfaces and reoriented so that the positive (upward) z-axis matches the main axes of the femur and tibia and the positive (forward) x-axis is defined based on the femur and patella. The final step in model generation is to reconstruct the patellar tendon attachment points to the tibia and patella in order to ensure good contact with no gaps. Because the MRI must be done with the patient lying prone, the patient's skin surface is deformed under gravity in a way that is not representative of the vertical orientation of the limb in a socket. To correct for this, a digital image correlation (DIC) system is used to capture accurate skin geometry while the limb is suspended vertically [3], and then the generated DIC skin surface is aligned with the MRI model of the bones using the MRI skin markers for alignment. In some embodiments, for the DIC imaging step, the limb is covered in white alcohol-based paint with a random black speckle pattern airbrushed on top. The DIC system uses cameras with overlapping fields of view to match speckle points between undeformed (straight) and deformed (bent) image sets. After all image sets are matched, a 3D point cloud is created. In some embodiments, the point cloud is imported into a software program (for example, Rhinoceros 5 3D modeling software), meshed, and any holes are patched and smoothed. The resulting surface is merged with the biomechanical model in replacement of the MRI skin surface. This constitutes the final biomechanical model.

The biomechanical model is imported, with all surfaces displayed. DIC skin is used in replacement of the MRI/CT skin if applicable. The model is then reoriented by finding the main z-axis of the tibia and rotating the entire model. The proximal end of the model is cut then flattened at a defined level. The default level is 10 mm below the most proximal point of the model. This flattened cut surface is then uniformly extruded proximally the same distance it was cut in order to preserve the total height of the model. The new model is then smoothed using, for example, a HC (Humphery's Classes) Laplacian smoothing method, which prevents shrinkage. The model is then reoriented based on a specified z-loading direction. The default loading algorithm uses the femoral center line as the basis for the load direction. A reference femur model is imported and scaled to approximately match the model femur size, and the entire model is reoriented so that the load line runs from the femoral head and through the center of the femoral condyles. Once this reorientation is complete, the top surface is again cut, flattened, and extruded to be flat with the XY-plane. This finishes the initial model processing.

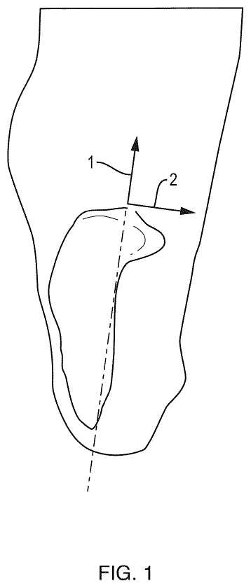

In order to best match the socket geometry and alignment to a subject's residual limb, the axes to define the cutline differ from the global coordinate system and are specified based on the following. First the longitudinal directional axis of the tibia is calculated using the singular value decomposition of the matrix of tibia vertex coordinates. This is referred to as the tibia local Z-axis, labeled as 1 in FIG. 1. The tibia local Y-axis is parallel to the global y-axis, that is a vector of direction [0 1 0] and is perpendicular to the plane of the page in FIG. 1. The tibia local X-axis is defined as the cross product between the local Z-axis and local Y-axis and is labeled 2 in FIG. 1. The alignment of the cutline definition axis with the tibia direction allows for improved placement of cutline points relative to the residuum.

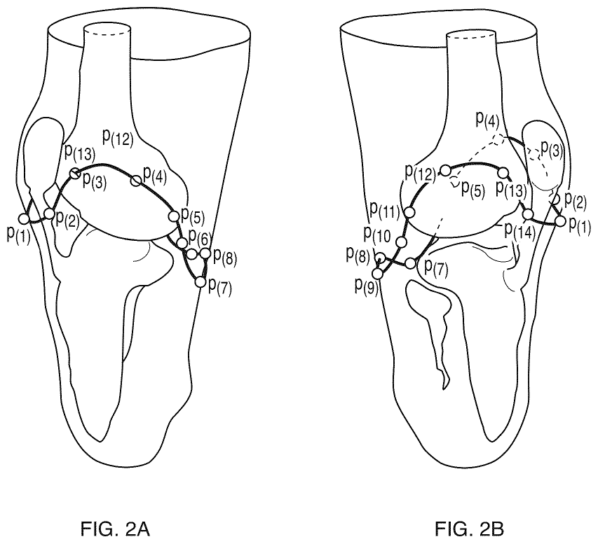

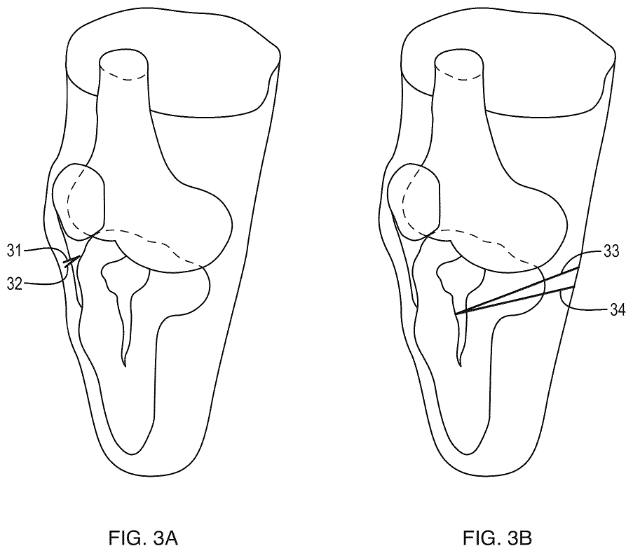

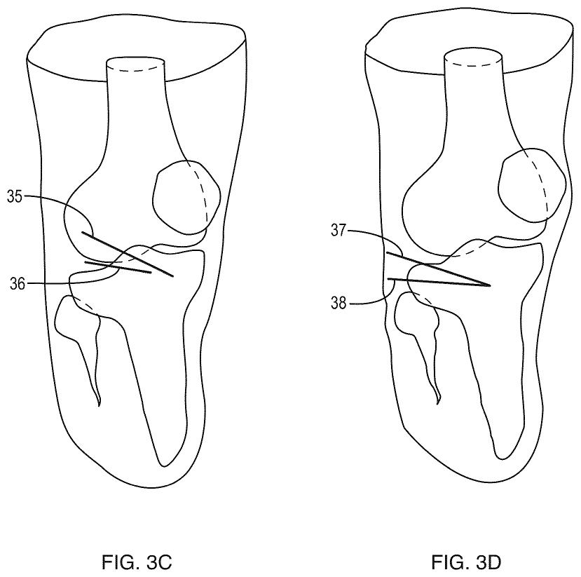

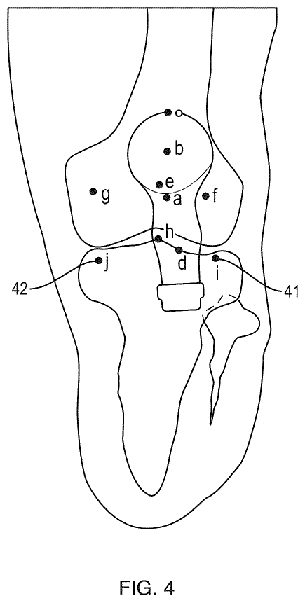

The positions of the 14 cutline control points, shown in FIG. 2, are calculated based on anatomical locations and ray tracing parallel to the tibia local axes to find corresponding points on the skin surface. Tibia local axes will also be referred to as local axes for conciseness. Ray tracing involves defining a vector in the desired direction, in this case parallel to the local X-axis, and using the Möller-Trumbore intersection algorithm to determine which faces on the triangular skin mesh the ray intersects. This allows accurate mapping of skin surface points from anatomical locations. Points are always mapped sequentially starting from the patellar tendon in a counterclockwise direction when viewed from the proximal end of the residuum. This is independent of amputation side. Because the tibia local coordinate system has redefined X and Z-axes but shares a Y-axis with the global coordinate system, the points whose ray tracing direction lines are affected by this change in axis (1, 7, 8, 9) are shown in FIG. 2. The position of Point 1 is found by locating the centroid of the patella, adjusting the z-coordinate to be between the middle of the patellar tendon and the bottom of the patella, and then ray tracing along the negative tibia local X-axis to place the point on the skin (FIG. 3a). Label 31 shows the ray tracing direction vector based on the global coordinate axes and label 32 shows the altered ray tracing direction vector based on the local coordinate axes. Point 2 is defined based on a ray direction parallel to the negative local Y-axis and an origin with the same X and Y coordinates as the centroid of the patella but shifted downward in Z. Point 3 is defined by a ray direction parallel to the negative local Y-axis and an origin based off the centroid of the patella but shifted posteriorly in X and proximally in Z. Point 4 is defined by a ray direction parallel to the negative local Y-axis and an origin based off the geometric center point of the femoral head. Points 5 and 6 are also defined by a ray direction parallel to the negative local Y-axis and origins based on the nearest tibia condyle (medial if right limb, lateral if left limb) but shifted posteriorly and distally. The ray tracing origin of Point 7 is located at the X-position of the middle of the patellar tendon, Y-position of the inner bottom tibial condyle, and Z-position of the average between the Z-positions of the bottom of the tibial condyles (Points i and j in FIG. 4, labeled 41 and 42 respectively). The position of Point 7 is then found by ray tracing along the direction of the positive tibia local X-axis (FIG. 3b). Label 33 shows the ray tracing direction vector based on the global coordinate axes and label 34 shows the vector based on the tibial coordinate axes. The ray tracing origin of Point 8 is located at the X and Y position of the patellar tendon, and Z-position of the average between the Z-positions of the bottom of the inner and outer condyles. The position of Point 8 is then found by ray tracing along the direction of the positive tibia local X-axis (FIG. 3c). Label 35 shows the ray tracing direction vector based on the global coordinate axes and label 36 shows the vector based on the tibial coordinate axes. The ray tracing origin of Point 9 is located at the X-position of the middle of the patellar tendon, Y-position of the outer bottom tibial condyle, and Z-position of the average between the Z-positions of the bottom of the inner and outer condyles. The position of Point 9 is then found by ray tracing along the direction of the positive tibia local X-axis (FIG. 3d). Label 37 shows the ray tracing direction vector based on the global coordinate axes and label 38 shows the vector based on the tibial coordinate axes. Points 10 and 11 are defined by a ray direction parallel to the positive local Y-axis and an origin based off the outer tibia condyle (lateral if right limb, medial if left limb). Points 12-14 are analogous to points 4, 3, and 2 respectively, but with a ray direction parallel to the positive local Y-axis so they are on the opposite side of the residuum.

Subjects with shorter residuums often experience medial-lateral instability when wearing sockets generated without default algorithm. To correct for this, the medial/lateral lobes of the cutline are raised to provide extra stability. The lobes are defined by points 2-5 on the medial side and points 11-14 on the lateral side, as shown in FIG. 2. Point 2 is analogous to point 14, point 3 to point 13, point 4 to point 12, and point 5 to point 11. Each point can be adjusted individually. For example, on one subject points 2 and 14 were raised 10 mm, points 3 and 13 by 30 mm, points 4 and 12 by 30 mm, and points 5 and 11 by 35 mm. If the raised points caused the cutline shape to deform too much, points 6 and 10 could be moved in the anterior/posterior direction to smooth out and sharp transitions.

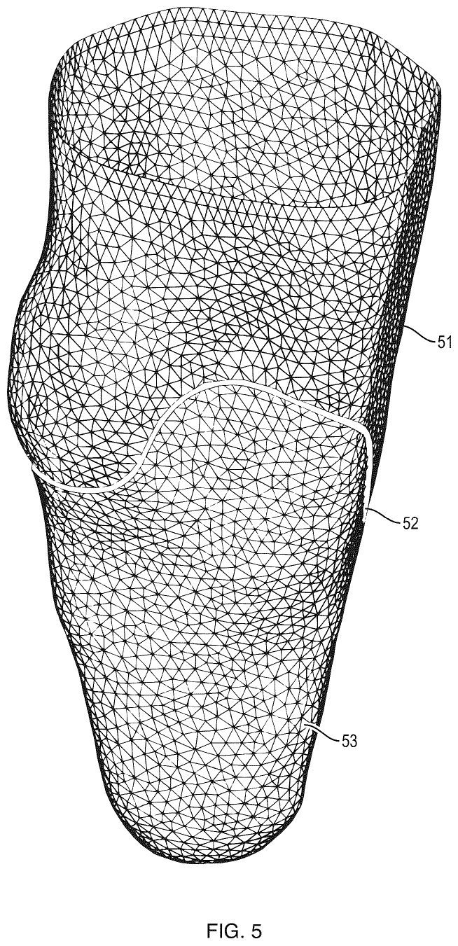

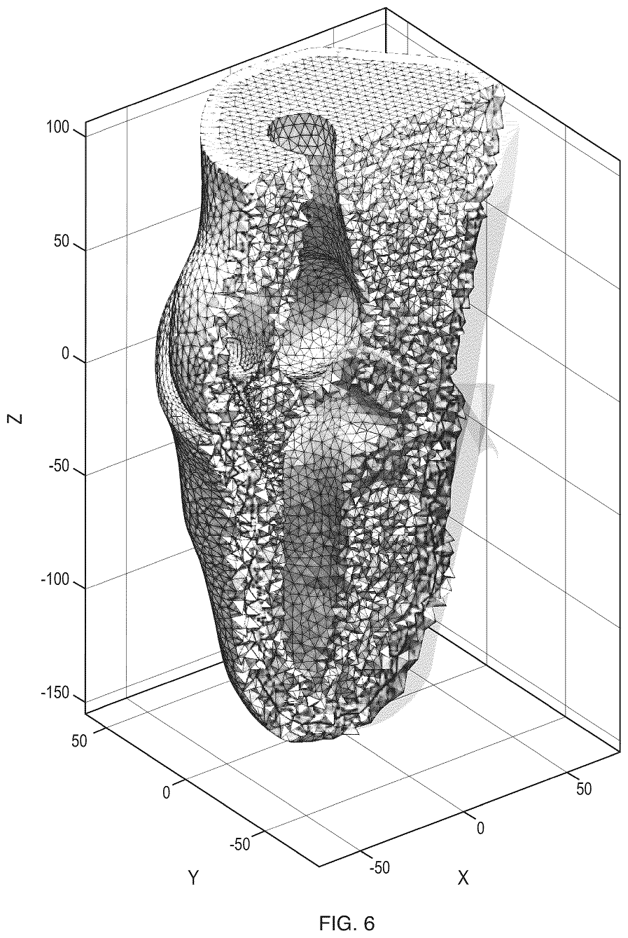

The cutline is defined on the socket by ray tracing a composite mesh curve on the skin surface of the model that intersects all 14 points. This curve is first approximated by finding all the lines on the model triangular mesh that are closest to the wanted curve and that form a closed loop. These lines are then grouped, assimilated, and smoothed until they form a continuous spline that is the socket cutline. The faces and nodes around the spline are repaired so there are no holes in the mesh. FIG. 5 shows the final geometrical regions. The portion of the model above the curve, labeled 51, is the proximal region and only the liner is offset from the skin. The portion of the model below the curve, labeled 53, is the socket region and both the liner and socket are offset from the skin. The cutline is labeled 52. The socket and liner surfaces are offset from the skin based on predefined thickness. The liner inner and outer surfaces are capped with a flat mesh at the top of the model. The socket inner and outer surfaces are capped by creating a lofted surface between the inner and outer top curves of the surfaces. The profile of this lofted surface is a sine curve connecting the inner and outer socket surfaces that is traced circumferentially along the cutline to enclose the top edge gap of the socket and create a unified body. The entire model is then meshed in 3D (for example using TetGen™) to create a solid body mesh, shown in FIG. 6, that can be used as the model input for finite element analysis.

The next step in the design process simulates donning and loading scenarios by running finite element analysis on the mesh residuum+liner+socket model. The mesh model from the previous step is imported and surface and geometrical features are extracted. The boundary conditions are set: Bones are fully supported so they have no volume change; The top surface is supported in the z-direction as a fixed end condition, and the skin surface is designated as the surface to which pressure is applied.

For this particular example, finite element analysis (FEA) is performed through a package called FEBio™. FEA parameters are set such as: maximum and minimum time step size, number of time steps desired, max number of reformations, optimum number of retries, etc. The FEA parameters can be changed if a model does not converge.

In some embodiments, each iteration of the FEA process comprises of 5 steps. First, liner fitting pressures, described below, are applied to the biomechanical model. This is done until the pressure at the skin interface reaches specified liner fitting pressure thresholds. Once the spatially-varying liner fitting pressures are applied to the biomechanical model, the second step involves attaching the liner material layer to the biomechanical model, and then applying its material properties. Following this procedure, the liner fitting pressures are then ramped down until they are eliminated, causing the modeled tissues to expand against the liner, stretching the compliant liner material as determined by FEA simulation. This process simulates the computational donning of the liner. In the third step, pressures are applied to the liner and biomechanical model until the pressures reach the socket fitting pressure thresholds. The deformed liner+tissue geometry caused by the socket fitting pressures defines the socket equilibrium geometry. The fourth step involves creating the socket, applying its material properties, and then ramping socket fitting pressures down to simulate donning of the socket. Because in this example the socket is rigid, there is no change in tissue+liner shape during this step. The fifth and final step is applying body weight to the entire model to simulate standing load in a method called force control, described below.

Several iterations of FEA are run for full socket generation, with the output from the last iteration imported as the input for the next. Most typically, three iterations are performed, as this number was determined to give the best geometrical fit without taking an unreasonable amount of time. As discussed below, one iteration is run first to generate the liner geometry. The liner is then printed, measured to find the actual thickness, and a full three iterations of FEA are run to generate the socket. The liner needs fewer iterations of FEA because it is made of compliant material, so small deviations in geometry are less consequential than they are on the socket.

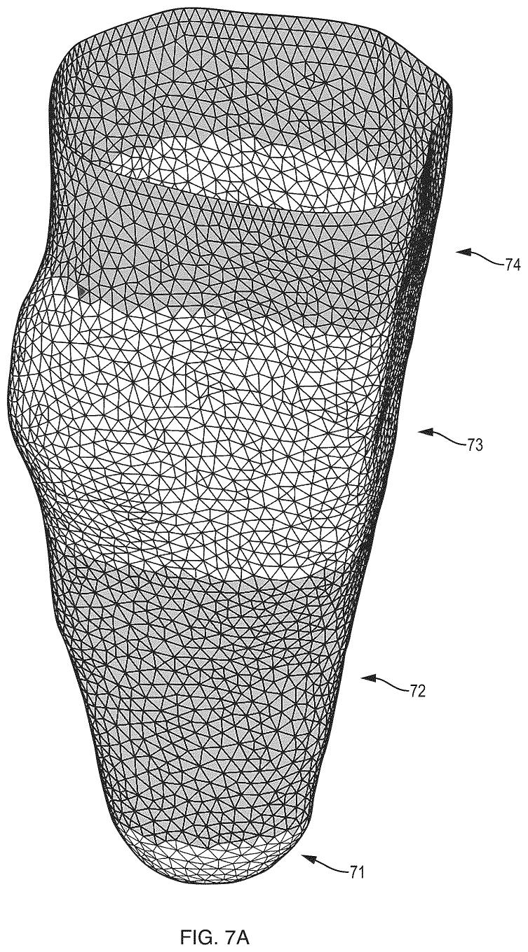

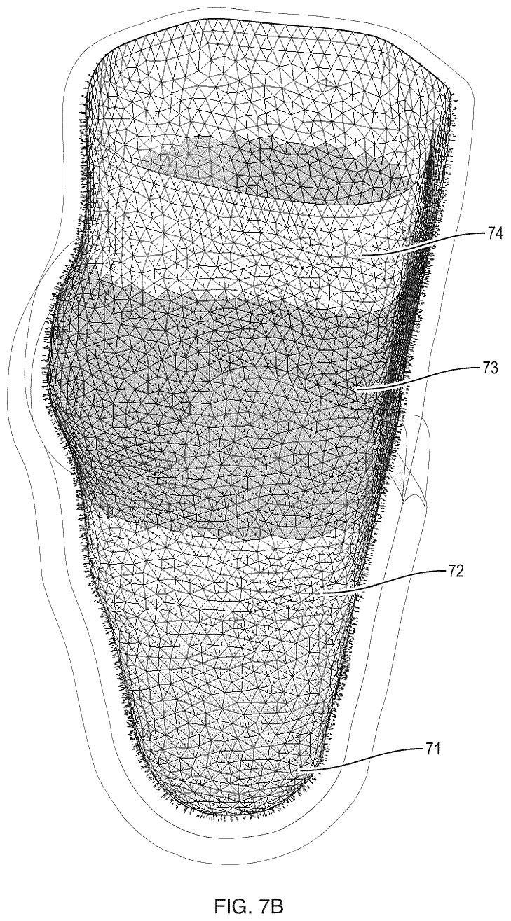

In order to improve the fit and comfort of the liner, in some embodiments the algorithm divides the amputated residuum into a spatially-varying pressure map with a plurality of regions of distinct pressure. This map is used in finite element analysis during liner design and donning. In some embodiments for a transtibial amputation, four regions are used, namely the distal end, lower leg, knee, and upper leg, all shown in FIGS. 7A, 7B. It should be clear to one of ordinary skill in the art that the geometric region definitions, as well as the associated pressure values, could be varied to optimize comfort and liner performance across individual users. For example, for subjects with abnormally short or long residuums, the distances used in the region definitions, as well as each region's pressure values, can be altered to accommodate individual subject needs. Each region has a fitting pressure defined as a multiple of the base pressure of x=15 kPa, as that is the target mean skin surface pressure after liner donning under no load [4]. The most distal of these is the distal end, defined as all points below the minimum point of the tibia in the z-direction (vertical). This is region 71 in FIGS. 7A, 7B. Pressure in this region is set to the lowest value of all the liner regions at 3.5×, or 52.5 kPa. Moving up the residuum, the next region is the lower leg region, defined as all points between the minimum point of the tibia and 20 mm below the maximum point of the tibia in the z-direction. Pressure in this region is allowed to vary, increasing from 3.5× (52.5 kPa) distally to 6.5× (97.5 kPa) proximally. This provides a smooth transition region between the sensitive distal end of the residuum and the load-bearing patellar and knee region. This is region 72 in FIGS. 7A, 7B. The upper leg region is defined as all points more proximal than 70 mm above the maximum point of the tibia in the z-direction. The upper leg is not load bearing as it is proximal to the socket cutline, and fitting pressure is set to 6.5× in order for the liner to sufficiently attach to the limb. This is region 74 in FIGS. 7A, 7B. Finally, the knee region is all points not included in the other three regions, or generally between 20 mm below the top of the tibia to 70 mm above the top of the tibia (top of the lower leg region to bottom of the upper leg region). Due to the knee region being less sensitive to pressure and the patellar tendon being one of the primary load bearing parts of the residuum, liner fitting pressure here is set to the highest of the liner fitting pressures at 7.5× (112.5 kPa). This is region 73 in FIGS. 7A, 7B. In addition, high liner fitting pressure around the knee is needed in order to prevent discomfort posterior to the knee during knee flexion. Higher fitting pressure allows the liner to better conform to the skin, preventing excessive material bunch-up when the knee is bent. In various embodiments, the spatially-vary pressure map techniques discussed herein are applied to designing liners that are configured to be applied at other biological body segments, for example an elbow region.

This spatially-varying pressure field is applied to the biomechanical model during finite element analysis to design the liner geometry and simulate liner donning. Pressure is applied to the skin outer surface, deforming the residuum based on soft tissue properties as well as bone geometry. The pressure is increased across the residuum until the regions reach the threshold pressures defined by the spatially-varying map. During this process the limb soft tissue develops stresses while the liner remains stress-free. The deformed skin geometry defines the liner inner surface at equilibrium, i.e. before donning. After the threshold pressures are reached, the liner is given a specified thickness and properties of the material from which it will be fabricated. To simulate liner donning, the limb is allowed to relax into the liner as applied pressures are ramped down. The liner is made of a compliant material, so as applied pressure is ramped down the limb and liner expand. The liner now has physical properties and thus exhibits a stiffness and develops material stresses. The limb is allowed to relax into the liner until the system reaches a state of dynamic equilibrium.

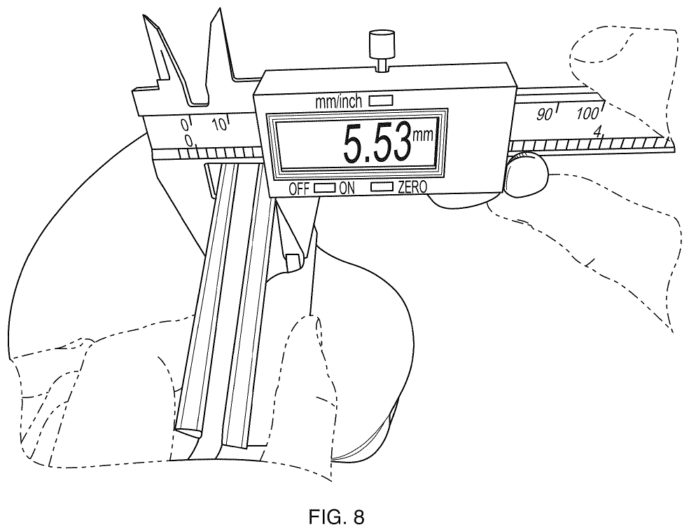

During experimental interface fittings, liners were generally printed at a nominal thickness of 7 mm. However, as will be understood by those of ordinary skill in the art, the liner thickness can be adjusted based upon individual subject needs. 3D printing of soft materials such as silicone is still in relative infancy, so the thickness of the actual liner often varies significantly. In order to correct for this variation within the design algorithm, the liner is designed, printed, and measured before running FEA to generate final socket geometry. Actual liner thickness is measured by sandwiching the liner between two rigid metal plates, finding the total thickness using calipers or a micrometer, and subtracting out the thickness of the plates. This is shown in FIG. 8. Measurements are taken at various points around the liner cuff and averaged to get a final value. This assumes that the distal end of the liner is approximately the same thickness as the cuff. The same process can be done by sandwiching the entire liner near the distal end and dividing the calculated total thickness by 2, but due to the taper of the liner towards the distal end this is unreliable as it is difficult to avoid folds when sandwiching the liner. Once the actual thickness of the liner is found, the model is re-meshed and the entire pipeline is rerun to generate final socket geometry. Once 3D printing technology improves, the liner can be printed at a specified thickness with high levels of repeatability and accuracy, negating the need to measure the liner prior to finalizing the socket computational design steps.

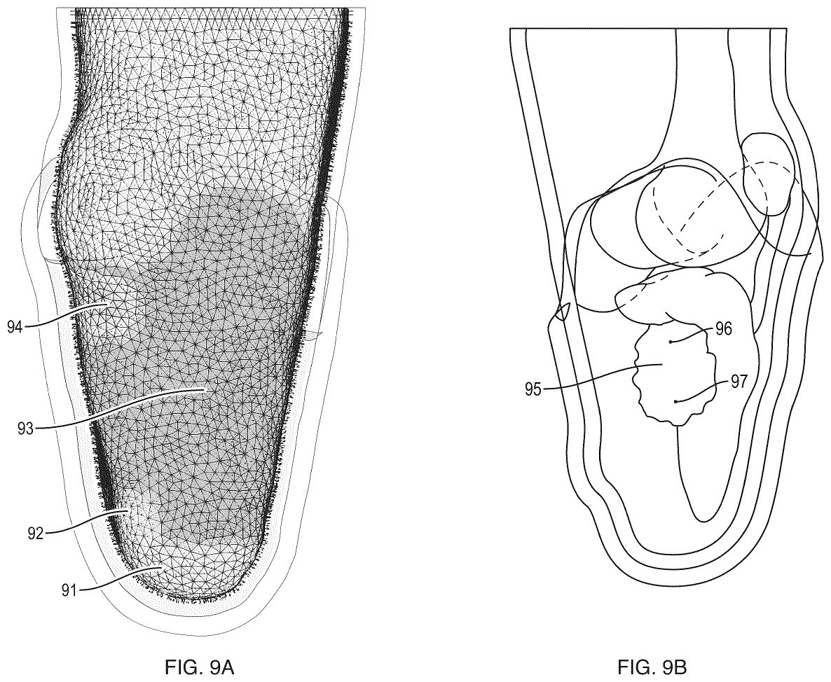

Socket fitting pressures are defined in a plurality of distinct anatomical regions aiming to alleviate sensitive regions. In one embodiment for a transtibial amputation, these regions are shown in FIG. 9, with the distal region labeled 91, residual region labeled 93, patellar region labeled 94, and fibular head region labeled 95. The distal pressure region is defined as everything below 1 cm above the distal point of the tibia. Pressure in this region is set to zero, which means the socket adds no additional pressure to the distal end of the residuum when donned. This aims to relieve pressure at the distal end of the tibia, which is the most common pain area. In addition, an extra window of zero socket fitting pressure is added around the anterior distal tibia, labeled 92 in FIG. 9. This window is defined to be 3 cm tall and 2 cm wide. It is positioned based on a tibia centerline vector, shown in FIG. 10. The window bottom is located at the minimum z-point of the tibia and its width is centered around the center line. The window can be expanded, shrunk, or removed based on subject preferences.

The other common sensitive region on a transtibial residuum is around the fibular head protuberance on the lateral side of the residuum. To accommodate this, a region of zero socket fitting pressure is defined around the fibular head. This region is an ellipse with one focus at the fibula head and the second 3 cm vertically below it, labelled 96 and 97 in FIG. 9. The ellipse is then defined as all points whose sum of distances to the foci is less than 5 cm. These default values give a region similar to the one labeled 95 in FIG. 9. The fibular head region is commonly altered to accommodate those with longer or more sensitive fibulas, and peroneal nerve areas distal to the fibula head, and this is done by changing the ellipse parameters.

Studies have shown that the patellar tendon is less sensitive to load than other regions of the transtibial leg, so we define a region of higher socket fitting pressure at the patellar tendon. This region is defined to be approximately the width of the patella and 5 cm tall, labeled 94 in FIG. 9. We find the geometric center of the patellar tendon and then center the pressure region 5 mm above this point. This is done because after long-term use, subjects noted excessive pressure on the top of the tibia rather than on the tendon. The patellar tendon region pressure is generally set to be 3.2× (48 kPa) of additional pressure on top of the liner fitting pressure. However, socket fitting pressures are more variable to subject preferences due to the relatively high-pressure tolerance in that region, and we have employed a range of patellar tendon pressures from 2.8× (42 kPa) to 4.2× (63 kPa).

Everything outside of the distal, fibular head, and patellar tendon regions is part of the residual fitting pressure region, labeled 93 in FIG. 9. This is the largest region and is critical for suspending the residuum in the socket. Default residual pressure is 1.2× (18 kPa) of socket pressure, although this is also variable from subject to subject. Generally, if a subject complains of excessive distal pressure, that typically means the residual pressure is not sufficiently large. Conversely, if a subject cannot get fully seated into the socket that means the residual pressure is too high and needs to be lowered. Based upon individual patient residuum biomechanics and fitting needs, residual pressures range from 0.6× (9 kPa) to 2.8 (42 kPa).

It will be clear to those of ordinary skill in the art that one can geometrically modify all socket fitting pressure regions, as well as to modify the target pressures. Further, one can add additional regions, if necessary, based upon subject preferences. For example, in a previous study, one subject had a sensitive scar on their anterior tibia crest between the distal and patellar regions, so an additional region of zero fitting pressure was added around this scar region.

The proposed algorithm employs a force control rather than a displacement control algorithm during finite element analysis loading simulation. Force control is the preferred method as an exact measured body weight can be used, removing error and producing more accurate displacements along the limb. The differences between force and displacement are as follows:

Displacement control: Apply a displacement value (e.g., 3 mm) to the nodes, then compute the reaction force of each node. Accumulate these reaction forces in the z-direction, to get a computed body weight. If the computed body weight is less than/greater than the real body weight, increase/decrease the applied displacement, until the computed body weight is equal to the real body weight (or the error is within a range that is acceptable).

Force control: Distribute the real body weight over the nodes, then compute the displacement generated in the z-direction. In this method, the body weight is always the actual body weight of the patient, and there is no guessing at what displacement is needed. This is a more realistic loading scenario compared to displacement control. It also provides benefits in repeatability, as if a subject gains or loses weight the body weight value can simply be changed as opposed to having to guess displacement values that correspond to the change in weight. However, because the reaction forces and displacements are calculated at every node, this method is more complex to implement, takes more computational time, and diverges more easily.

Once the displacement has been calculated using force control, it is possible to back-drive the model using displacement control as it should output approximately the right body weight. This may be useful if multiple socket variations of the same fitting pressures are needed quickly, as displacement control is computationally faster than force control.

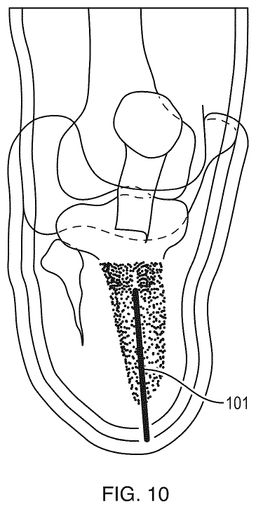

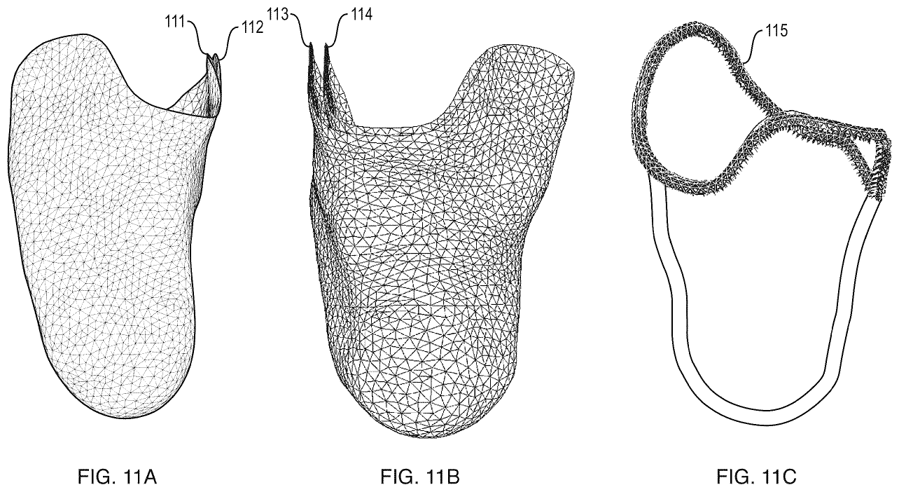

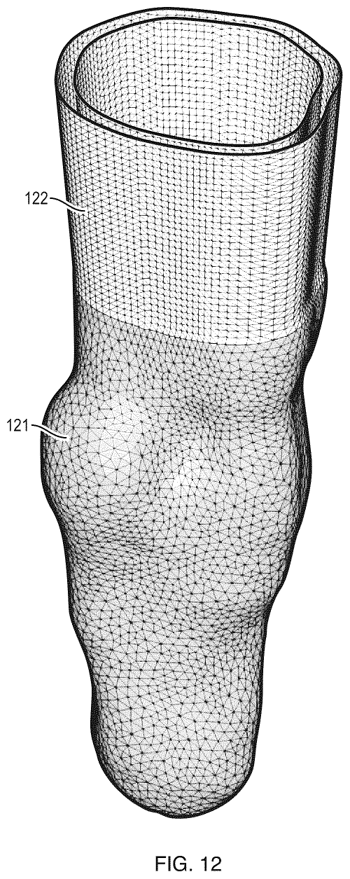

In the next step of the design process, further refinements are made to the socket and liner designs that either could not be addressed through the FEA simulation or are corrections for artifacts from FEA. The output from finite element analysis is imported. The top edge of the socket inner surface is mapped, and the surfaces of the medial lateral socket lobes are modified to correct for excessive inward curvature from FEA. This curvature can cause discomfort and make the socket difficult to don, so correction of this is important to socket comfort. FIG. 11A shows this correction, with the original inward curvature from FEA labeled 111 and the corrected geometry labeled 112. Regions for manual modification are defined and nodes are pulled out a specified amount. The socket inner surface is smoothed, and the final surface is then duplicated and offset by a specified socket thickness amount to create the outer surface. This offset is also shown in FIG. 11B as the gap between the inner surface, labeled 114, and the outer surface, labeled 113. Socket thickness can be varied based on the expected application of the socket. If the socket is being used only for geometric fit testing without standing or walking, the thickness can be reduced in order to save printing time and costs. If the socket will be used as a proper check socket, a thickness of 5 mm or greater can be used. The two surfaces are unified at the top edge by a lofted surface of the same definition as earlier in the framework, labeled 115 in FIG. 11C. The liner is then defined, and the top curve is extruded upward in the Z-direction to reach the subject's mid-thigh level. This extrusion 122 is combined with the rest of the liner 121 and smoothed, giving the final liner geometry shown in FIG. 12. If saving is on, the final socket and liner meshes are exported (for example, as STL files) for 3D printing.

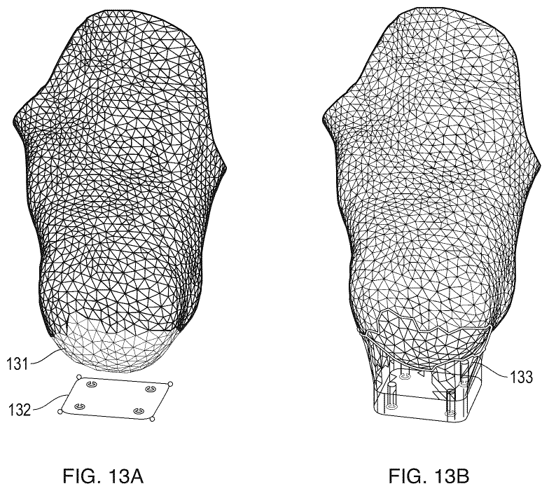

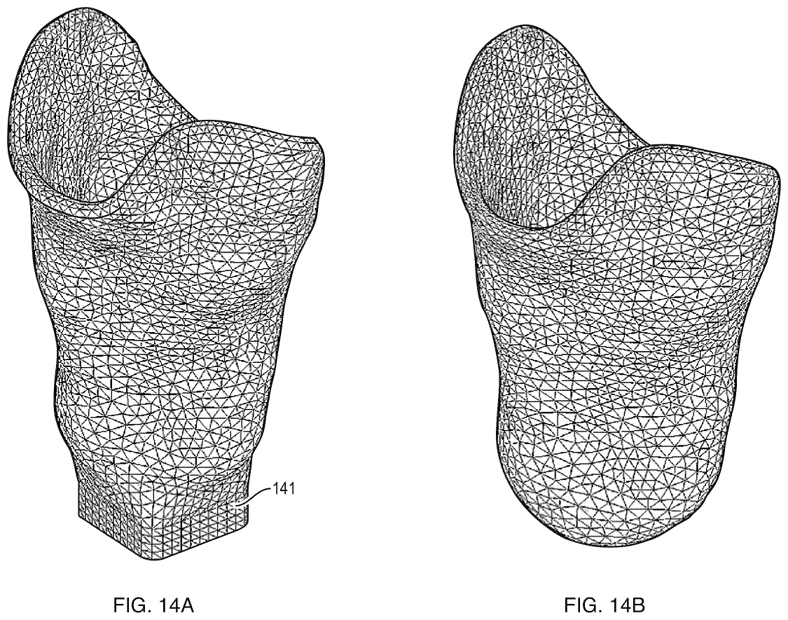

In various embodiments, a control has been added to include/exclude a socket foot attachment part. This attachment allows for an integrated connection point to an alignment pyramid that eliminates the need for a prosthetist to align the socket with the foot/ankle device. If excluded, the socket distal end can be unchanged from the model. If included, a rectangular attachment segment can be added to the distal end of the socket. The pyramid attachment should only be added if proper alignment measurements are known and input into the code. This attachment is positioned relative to the socket based on the load line imported from FEA results. Four points are defined based on defined dimensional parameters so that the load line runs through the center of the foot. A curve 132 that defines the bottom edge of the foot is drawn based on these four points, as well as four circles for tapping holes that can allow for connection to a socket pyramid. The points and curves are shown in FIG. 13A. The closest faces and nodes at the distal end of the socket 131 are found and then a surface 133 is extruded from the bottom curve 132 to the faces and nodes 131, shown in FIG. 13B. This surface is then meshed and merged with the socket mesh to create the enclosed foot 141. Comparison of foot/no foot sockets is shown in FIGS. 14A-14B. For this associated clinical trial, the foot was excluded on all sockets and proper alignment and attachment was done by a professional prosthetist.

Not all pain points can be sufficiently addressed through the FEA optimization, especially for subjects with particularly sensitive residuums. Even at areas of zero additional socket fitting pressure, the socket may cause pressure during motion due to movement of the limb relative to the rigid socket. Often problematic regions are not known until the first check socket design has been donned and tested in walking.

In order to eliminate pain regions, we first look at the final FEA socket fitting pressure in that area. If it is high, a new low-pressure region can be created around the pain point and FEA can be rerun in order to optimize the new socket. If the fitting pressure is already zero after FEA, we have the ability to manually move the mesh nodes away from the liner and limb in order to create more room to relieve pain. The selection of a region to adjust nodes has been achieved in several ways:

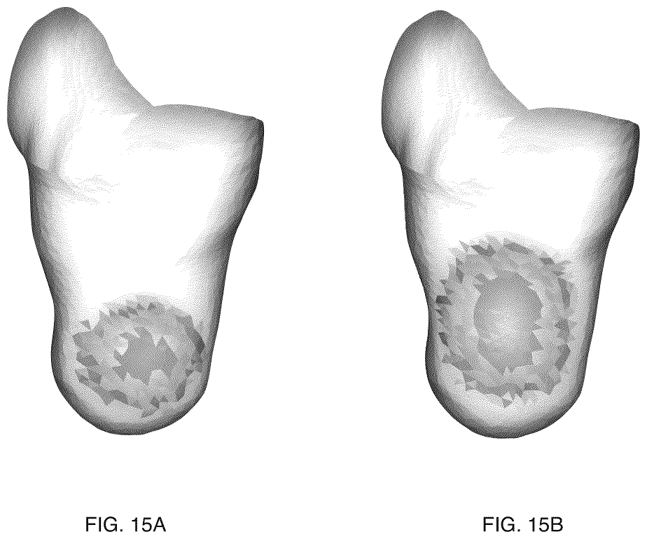

The first involves selecting a node to center around and creates a circular region of specified radius around this node. The edges of the region are then smoothed so that the pulled-out area is smoothly integrated into the rest of the socket. An example is shown in FIG. 15A. In this case, the nodes around the center are moved away from liner and limb 3 mm and the edges are tapered down 2 mm or 1 mm to integrate them into the socket without harsh boundaries.

The second is similar to the first, except two nodes are chosen to be the foci of an elliptical region. This gives more flexibility to accommodate larger oblong sensitive areas. An example is shown in FIG. 15B.

Other methods of manipulating mesh nodes to modify interface geometry are possible, such as polygonal regions based on the number of defined points.

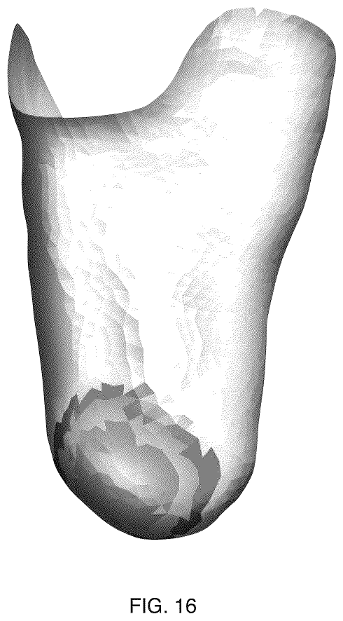

Due to common complaints of pain at the distal end of the tibia, even at zero fitting pressure, nodes around this area are pulled out 3 mm in a circular region of radius 20 mm at the anterior distal end of the socket for all subjects. This is shown in FIG. 16.

Mesh modification can also be automated based on the target pressure map at FEA input and the final pressure map at FEA output. An algorithm is demonstrated that finds discrepancies between target and resultant pressures on the socket pressure map and adjusts nodes in regions where the discrepancy is larger than a specified threshold. This can be seen as a way to correct for inadequacies in the finite element analysis simulation.

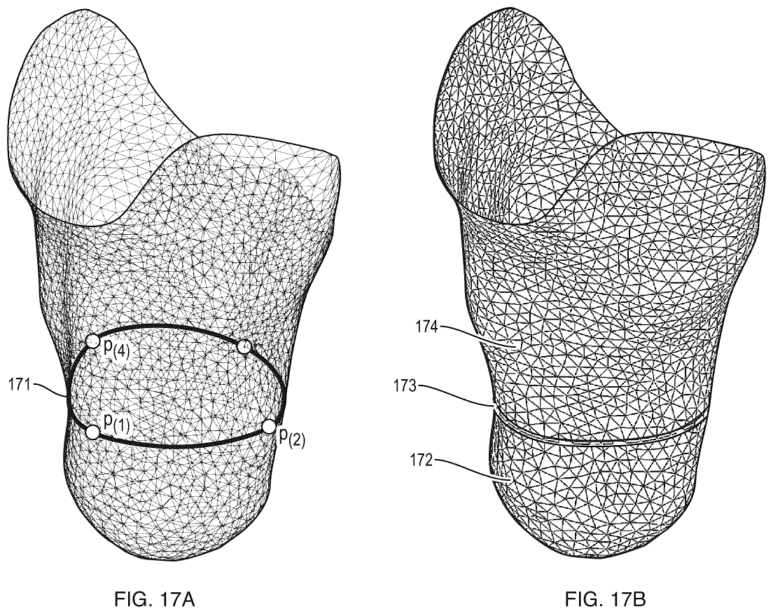

It has been found that different smoothing algorithms are occasionally needed for different patients based on limb sensitivity. This is because there is a tradeoff between smoothing types, as some provide better surface quality but create larger changes in dimension, especially at areas of high curvature. A method to define distinct regions along a mesh model allows smoothing to be done differentially in case different regions of the socket require different smoothing algorithms. The distal/proximal divide z-level is defined as the top of the added anterior distal window of zero fitting pressure. Four points at approximately this z-level (with buffer of ±2 mm) are found, one each at the anterior, medial, posterior, and lateral sides of the socket. A path that intersects all four of the points is found using ray tracing, and then the path is smoothed to a straight line 171 by altering the mesh. This process is shown in FIG. 17A. The final regions are shown in FIG. 17B. The transition region 173 around the straight dividing line is smoothed using a modified Laplacian algorithm so that there is not a jump shift between proximal 174 and distal regions 172.

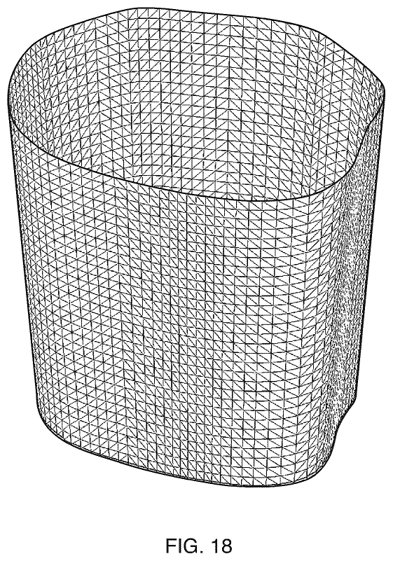

Prosthetic liners normally extend to mid-thigh level for comfort and security. Imaging is only taken to just above the knee proximally, so the top curve of the liner is extruded to reach the mid-thigh level. The distance of the extrusion is determined by the subject's femoral length. Previously, this was done as a straight vertical extrude, but that was eliciting complaints of tightness from several subjects, as the mid-thigh circumference is usually larger than above-knee circumference. The modified method flares the extrusion out based on circumference measurements of the subject's thigh. The flare is a linear mapping from the top curve of the liner to the mid-thigh circumference, and the extruded portion is constant thickness. This is shown in FIG. 18.

The prosthetic liner, while made of compliant silicone, often causes skin irritation and pain during knee flexion due to shear forces between the liner and the skin. In addition, material will bunch up posterior to the knee during knee flexion as the distance between points on the leg is reduced. To improve comfort, it is necessary to reduce the amount of material in this area. This can be done by reducing thickness, which also reduces the shear force the liner applies to the residuum for a given amount of knee flexion. Commercial liners are often either uniform thickness or slightly thinner posteriorly in order to reduce the aforementioned bunching when the knee is bent. In various example embodiments, custom liners discussed herein are fabricated at a nominal thickness within the range 5 mm to 7 mm. To address these concerns, an algorithm to create liners that aim to reduce discomfort at the knee during flexion by varying the thickness was developed.

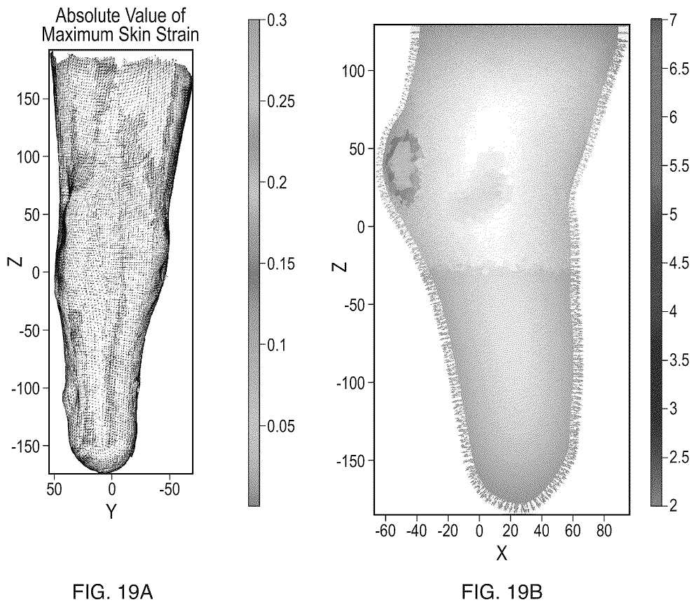

The algorithm defines the liner thickness as inversely proportional to the absolute value of skin strain for areas above the socket cutline. The area below the socket cutline, therefore contained in the socket, are kept relatively thick as they are load-bearing. First (compressive) and second (tensile) strain values are calculated for the skin based on 3D digital image correlation (DIC) of the knee in flexion. Using the DIC data, a triangular mesh model is created of the limb. Each strain value in the array corresponds to one triangular face in the mesh. A maximum strain threshold is set to 0.3 to remove any outlying data points in the strain array. FIG. 19A shows the absolute strain values mapped on to the unprocessed DIC model of the limb. To correctly map strain values to a thickness, strain values can first be assigned to each vertex in the array since these are the points that are offset to create the outer liner surface. For each vertex in the array, the function can average the strain values of all adjacent faces and assign this average value to the vertex. Once each vertex has a value, the strain array is normalized from 0 to 1. Then the maximum strain values are mapped to a minimum thickness of 2 mm and the minimum strain values are mapped to a maximum thickness of 7 mm. FIG. 19B shows the variable liner thickness offset from the skin, with constant thickness below the socket cutline.

The algorithm is implemented after FEA is run to determine final socket and liner geometry. Because the thickness is only varied around the knee proximal to the socket, this does not change the fit of the socket. Liner thickness below the cutline is kept at a uniform thickness (e.g., 7 mm). After the variable thickness algorithm is implemented, the final liner is smoothed so that there is no step boundary between regions. As an alternative liner embodiment, the liner could be made thin above the socket cutline (e.g., 2 mm) but uniform without any correlation to underlying skin strain, enabling the knee to flex and extend while minimizing liner-skin shear forces.

After all changes are made to the prosthetic interface, files are prepared for 3D printing. Mesh models for liner and socket are exported as, for example, Standard Tessellation Language (STL) files, the most common file format for 3D printing. In various example embodiments, the STL files are imported then renamed and exported to a separate directory labeled by the date. In various embodiments, multiple STLs can be processed at once. In various embodiments, no modifications are made to the socket geometry.

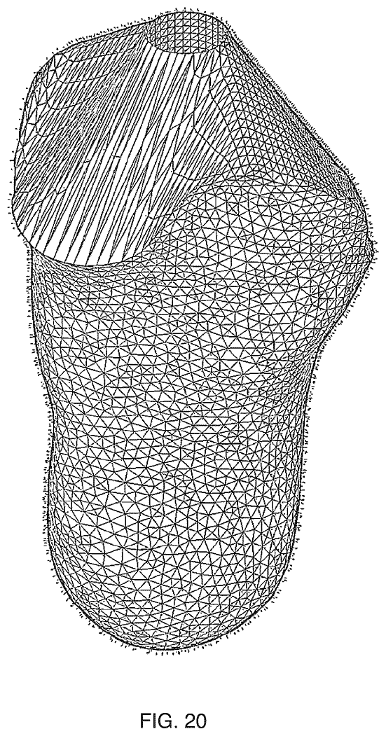

If a socket is determined to be comfortable and a permanent socket is wanted, a positive mold is needed for carbon fiber or fiberglass wrapping. The framework has the capability to design a positive mold for a permanent socket. The socket inner surface is taken to be the outer surface of the positive. This surface is then offset inward a specified amount to create a hollow solid. The reason this is done is to save material during fabrication. A circular space is defined as a cylinder in the Z-direction that provides a way for the positive to be held for wrapping. This cylinder is aligned with the center axis of the socket. If the socket has a foot attachment, the center axis is taken as the center axis of the attachment. If the socket does not have a foot attachment, the center axis is determined based on the socket load line in the same way the foot attachment placement would be calculated. The body is enclosed by lofting a surface between the top curve of this cylinder and the socket cutline, shown in FIG. 20. The body is then checked for holes and intersecting surfaces. In various embodiments, if the checks are passed, the final positive is saved as an STL file.

In order to fabricate custom liners rapidly and with high precision, a new manufacturing method is needed to replace silicone casting. Novel advances have been made in soft material 3D printing, allowing printing of custom liners from platinum-cure silicone or polyurethane. This eliminates the mold-making step used in casting while retaining the ability to produce geometrically accurate compliant liners. This technology provides benefits in both fabrication time and material waste, especially if multiple liners are needed. Material properties for the silicone are measured via tension and compression tests, and characterization is based on an Ogden model. Properties are listed below:
Bulk modulus: 34.5 MPa; C1: 0.108 MPa; M1: −3.1; C2: 0.00168 MPa; M2: −0.746; Durometer: Shore 00-53.

The silicone printing process used, called Rapid Liquid Printing, is direct-write, or extrusion based. The 3D model is turned into a geometrical shell, and a toolpath is defined to create that shell. The thickness of the part is determined by the toolpath parameters. The process can be simplified by using general templates for repeat parts. The silicone is cured during the printing process, reducing post-processing steps. These templates allow for certain assumptions about a category of part such as liners such as size and thickness, reducing the processing and printing time. The extrusion rate for silicone printing is 12 ml/min. In various embodiments, on average our custom liners are 700-800 ml, giving an average printing time of around 1 hour.

Check sockets are also fabricated through 3D printing. The sockets are printed out of PCTG, an impact resistant and easily printable polyester compound. These sockets are uniform compliance and uniform 8 mm thickness. Printing times for the sockets depends on the size and thickness, but average times are around 6 hours. Both liner and socket arrive ready-to-wear.

3D printing allows for unique advantages during socket fitting and iteration. Because the entire design process and final STL model generation is contained in a single pipeline, multiple variations of the same general interface can be designed and fabricated simultaneously. This enables a patient to try multiple different sockets and liners and choose which variation best fits their limb. This is an advancement over the conventional process because rather than having only one check socket that must be taken off and modified for every area of concern, the patient gets a direct comparison between multiple designs. This is not reasonably possible in the conventional process because molds for the check socket are destroyed during the fabrication process.

### REFERENCES

[1] Hugh M. Herr, Kevin Mattheus Moerman, & David Moinina Sengeh. (2019). Method And System For Designing A Biomechanical Interface Contacting A Biological Body Segment (Patent No. US20190021880A1).
[2] Hugh M. Herr, Andrew Marecki, & David M. Sengeh. (2021). Variable Impedance Mechanical Interface (Patent No. US20210022891A1).
[3] Hugh M. Herr, Kevin Mattheus Moerman, Dana Solav, Bryan James Ranger, Rebecca Steinmeyer, Stephanie Lai Ku, Canan Dagdeviren, Matthew Carney, German A. Prieto-Gomez, Xiang Zhang, Jonathan Randall Fincke, Micha Feigin-Almon, Brian W. Anthony, Zixi Liu, Aaron Jaeger, & Xingbang Yang. (2021). Quantitative Design And Manufacturing Framework For A Biomechanical Interface Contacting A Biological Body Segment (Patent No. US20210145608A1).
[4] Moerman, K. M., Solav, D., Sengeh, D. M., & Herr, H. M. (2016). Automated and Data-driven Computational Design of Patient-Specific Biomechanical Interfaces. https://doi.org/https://doi.org/10.31224/osf.io/g8h9n
[5] Ziegler-Graham, K., Mackenzie, E. J., Ephraim, P. L., Travison, T. G., & Brookmeyer, R. (2008). Estimating the Prevalence of Limb Loss in the United States: 2005 to 2050. Archives of Physical Medicine and Rehabilitation, 89(3), 422-429. https://doi org/10.1016/j.apmr.2007.11.00S
[6] Amputee Statistics You Ought to Know. (2012). https://advancedamputees.com/amputee-statistics-you-ought-know
[7] Limb Loss Statistics-Amputee Coalition. (n.d.). https://www.amputee-coalition.org/resources/limb-loss-statistics/
[8] Lofters, A., Guilcher, S., Maulkhan, N., Milligan, J., & Lee, J. (2016). Patients living with disabilities: The need for high-quality primary care. Canadian Family Physician Medecin de Famille Canadien, 62(8), e457-64.
[9] Sabzi Sarvestani, A., & Taheri Azam, A. (2013). Amputation: a ten-year survey. Trauma Monthly, 18(3), 126-129. https://doi.org/10.5812/traumamon.11693
[10] Seidell, J. C. (2000). Obesity, insulin resistance and diabetes—a worldwide epidemic. British Journal of Nutrition, 83(S1), S5-S8. https://doi.org/10.1017/S000711450000088X
[11] Geiss, L. S., Li, Y., Hora, I., Albright, A., Rolka, D., & Gregg, E. W. (2019). Resurgence of Diabetes-Related Nontraumatic Lower-Extremity Amputation in the Young and Middle-Aged Adult U.S. Population. Diabetes Care, 42(1), 50-54. https://doi.org/10.2337/dc18-1380
[12] How to Make Prosthetics. (n.d.). https://www.scheckandsiress.com/blog/how-to-make-prosthetics/
[13] Sengeh, D. M., & Herr, H. (2013). A Variable-Impedance Prosthetic Socket for a Transtibial Amputee Designed from Magnetic Resonance Imaging Data. JPO Journal of Prosthetics and Orthotics, 25(3), 129-137. https://doi.org/10.1097/JPQ.0b013e31829be19c
[14] J. E. Sanders, E. L. Rogers, E. a Sorenson, G. S. Lee, and D. C. Abrahamson, “CAD/CAM transtibial prosthetic sockets from central fabrication facilities: how accurate are they?,” J. Rehabil. Res. Dev., vol. 44, no. 3, pp. 395-405, 2007.
[15] V. Y. Ma, L. Chan, and K. J. Carruthers, “Incidence, prevalence, costs, and impact on disability of common conditions requiring rehabilitation in the United States: stroke, spinal cord injury, traumatic brain injury, multiple sclerosis, osteoarthritis, rheumatoid arthritis, limb loss, and back pa,” Arch. Phys. Med. Rehabil., vol. 95, no. 5, p. 986-995.el, May 2014.
[16] R. Gailey, K. Allen, J. Castles, J. Kucharik, and M. Roeder, “Review of secondary physical conditions associated with lower-limb amputation and long-term prosthesis use.,” J. Rehabil. Res. Dev., vol. 45, no. 1, pp. 15-29, Jan. 2008.
[17] S. M. Tintle, J. J. Keeling, S. B. Shawen, J. A. Forsberg, and B. K. Potter, “Traumatic and trauma-related amputations: part I: general principles and lower-extremity amputations.,” J. Bone Joint Surg. Am., vol. 92, no. 17, pp. 2852-68, December 2010.
[18] The PandoFit Solution. (n.d.). Retrieved Apr. 14, 2022, from https://prosfit. com/solution
[19] M Moerman, K. (2018). GIBBON: The Geometry and Image-Based Bioengineering add-On. The Journal of Open Source Software, 3(22), 506. https://doi.org/10.21105/joss.00506

The teachings of all patents, published applications and references cited herein are incorporated by reference in their entirety.

The various features described herein can be used separately or in combination.

Additional descriptions of methods and devices for generating biomechanical interfaces can be found in US20190021880A1 [1], US20210022891A1 [2], and US20210145608A1 [3], the entire teachings of which are incorporated herein by reference.

While example embodiments have been particularly shown and described, it will be understood by those skilled in the art that various changes in form and details may be made therein without departing from the scope of the embodiments encompassed by the appended claims.

What is claimed is:

1. A liner configured to serve as an interface between an external surface of a biological body segment of a subject and a prosthetic socket, the biological body segment being amputated below a joint, wherein pressure applied by the liner to the biological body segment varies over the length of the liner with a maximum pressure applied at the joint and a lesser pressure applied below the joint.

2. The liner of claim 1 wherein the pressure applied by the liner is lower than at the joint both below and above the joint.

3. The liner of claim 1 wherein the joint is a knee.

4. The liner of claim 1, wherein the maximum pressure applied at the joint is greater than 97.5 kPa.

5. The liner of claim 1, wherein the lesser pressure varies between 52.5 kPa and 97.5 kPa.

6. The liner of claim 1, wherein the liner comprises a first thickness below a cutline and a second thickness above a cutline and the first thickness is greater than the second thickness.

7. The liner of claim 6, wherein the second thickness is between 2 mm and 7 mm.

8. The liner of claim 1, wherein the liner is fabricated using direct-write, soft material 3D printing.

9. The liner of claim 8, wherein the liner is fabricated using direct-write soft material 3D printing from platinum-cure silicone or polyurethane.

10. A method for designing a liner interfacing an external surface of a biological body segment amputated below a joint, comprising:
with a computer:
forming a liner design based, at least in part, on a model of the biological body segment; and
adjusting the liner design in response to a simulated fitting pressure, the simulated fitting pressure varying over the length of the liner design with a maximum simulated fitting pressure being applied at positions in the liner design configured to interface at the joint and a lesser simulated fitting pressure applied at positions in the liner design configured to interface below the joint.

11. The method of claim 10, wherein the simulated fitting pressure applied at positions in the liner design configured to interface above and below the joint is lower than the simulated fitting pressure applied at positions in the liner design configured to interface at the joint.

12. The method of claim 10, wherein the joint is a knee.

13. The method of claim 10, wherein the maximum simulated fitting pressure is greater than 97.5 kPa.

14. The method of claim 10, wherein the lesser simulated fitting pressure varies between 52.5 kPa and 97.5 kPa.

15. The method of claim 10, wherein the liner design comprises a first thickness below a cutline and a second thickness above a cutline and the first thickness is greater than the second thickness.

16. The method of claim 15, wherein the second thickness is between 2 mm and 7 mm.

17. The method of claim 10, further comprising fabricating the liner based on the liner design using direct-write, soft material 3D printing.

18. The method of claim 17, further comprising fabricating the liner based on the liner design using direct-write soft material 3D printing from platinum-cure silicone or polyurethane.

19. A system for designing a liner interfacing an external surface of a biological body segment amputated below a joint, comprising:
a computer configured to:
form a liner design based, at least in part, on a model of the biological body segment; and
adjust the liner design in response to a simulated fitting pressure, the simulated fitting pressure varying over the length of the liner design with a maximum simulated fitting pressure being applied at positions in the liner design configured to interface at the joint and a lesser simulated fitting pressure applied at positions in the liner design configured to interface below the joint.

20. A method for interfacing a liner with an external surface of a biological body segment amputated below a joint, comprising:
applying a pressure by the liner to the biological body segment, the pressure varying over the length of the liner with a maximum pressure applied at the joint and a lesser pressure applied below the joint.
